(19) United States
(12) Patent Application Publication
(10) Pub. No.: US 2016/0338595 A1
(43) Pub. Date: Nov. 24, 2016

(54) OBJECT INFORMATION ACQUIRING APPARATUS

(71) Applicant: CANON KABUSHIKI KAISHA, Tokyo (JP)

(72) Inventor: Jiro Tateyama, Yokohama-shi (JP)

(21) Appl. No.: 15/155,446

(22) Filed: May 16, 2016

(30) Foreign Application Priority Data
May 19, 2015 (JP) 2015-101931

(51) Int. Cl. A61B 5/00; G01N 21/17

(52) U.S. Cl. CPC A61B 5/0095; G01N 21/1702; A61B 2560/0214

(57) ABSTRACT

An object information acquiring apparatus is used which includes: a light source controller that controls radiation of light from a light source; a detector that detects an acoustic wave generated by an object irradiated with the light; an information processor that acquires characteristics information on an inside of the object using the acoustic wave; and a scan controller that changes a relative position between the detector and the object, this object information acquiring apparatus further including: a power source that supplies electricity for use by the object information acquiring apparatus; and a power source controller that controls activation timings for the light source controller and the scan controller so as to prevent a current value of a current flowing through the object information acquiring apparatus from exceeding an ampacity of the power source.

### BACKGROUND OF THE INVENTION

1. Field of the Invention

The present invention relates to an object information acquiring apparatus.

2. Description of the Related Art

Conventionally, ultrasound diagnostic apparatuses have been used for medical image diagnosis. The ultrasonic diagnostic apparatus transmits an ultrasonic wave to an object using a probe including a plurality of acoustic detection elements, and receives an echo reflected from a tissue boundary inside an object. As a result, morphological information on the inside of the object is acquired and can be utilized to discover a diseased segment such as a tumor. In recent years, in order to improve discovery efficiency for the diseased segment, attention has been paid to imaging of physiological information on the object, in other words, functional information on the object. As imaging means for the functional information, photoacoustic tomography (PAT) using pulsed light and ultrasonic waves has been proposed.

The photoacoustic tomography is a technique that utilizes a photoacoustic effect in which, upon absorbing light, a light absorber in the object expands and contracts to generate a photoacoustic wave, to image an internal tissue that is a source of the photoacoustic wave. A photoacoustic tomography apparatus irradiates the object with pulsed light to detect, at a plurality of positions, temporal changes in a photoacoustic wave generated by the object, using acoustic detection elements. The photoacoustic tomography apparatus then executes a reconstruction process that is a mathematic analysis process on reception signals to visualize information related to optical characteristic values for the inside of the object, thus obtaining three-dimensional images.

An apparatus in Japanese Patent Application Laid-open No. 2012-179348 includes an acoustic array detector provided such that reception surfaces of a plurality of acoustic detection elements each receiving a photoacoustic wave from the object are arranged at different angles. The apparatus further includes a scanner that moves the acoustic array detector in an X-axis direction, a Y-axis direction, and a Z-axis direction in order to move the relative position of a high-resolution region determined by the arrangement of the object and the acoustic array detector. The apparatus in Japanese Patent Application Laid-open No. 2012-179348 also uses a matching layer containing water or castor oil in order to acoustically couple the object and the acoustic array detector together.

Patent Literature 1: Japanese Patent Application Laid-open No. 2012-179348

### SUMMARY OF THE INVENTION

When the acoustic array detector is moved in the X-, Y-, and Z-axis directions as in the apparatus in Japanese Patent Application Laid-open No. 2012-179348, a scanner including a servo motor is needed. A circulator including a pump motor may also be provided in order to circulate the solution in the matching layer and to manage the liquid temperature of the solution. A temperature controller for liquid temperature management may be separately provided.

When the scanner or the circulator is powered on to start operation, an instantaneous flow of a current much larger than a current flowing in a steady state may be caused by an inductance component of the servo motor or the pump motor, leading to a transient current referred to as an inrush current and lasting only a short time. Furthermore, when a high-output pulse laser apparatus is used for a light irradiator that generates pulsed light, a power source has an increased capacity and thus an increased capacitance component. As a result, the inrush current at the time of power-on is increased.

When an allowable current value for a power source component such as a transformer or a circuit breaker is calculated, addition of all inrush currents flowing through the scanner, the circulator, the light irradiator, and the like results in a very large allowable current value. To achieve the large allowable current value, the power source component needs to be made larger, leading to the shortage of space and increased costs.

The present invention has been developed in view of the above-described problems. An object of the present invention is to provide an apparatus that acquires characteristics information on the inside of an object using photoacoustic waves and that adjusts inrush currents at the time of power-on to enable a reduction in ampacity of a power source.

The present invention provides an object information acquiring apparatus comprising:

alight source controller that controls radiation of light from a light source;

a detector that detects an acoustic wave generated by an object irradiated with the light;

an information processor that acquires characteristics information on an inside of the object using the acoustic wave;

a scan controller that changes a relative position between the detector and the object,

a power source that supplies electricity for use by the object information acquiring apparatus; and

a power source controller that controls activation timings for the light source controller and the scan controller so as to prevent a current value of a current flowing through the object information acquiring apparatus from exceeding an ampacity of the power source.

According the present invention, an apparatus can be provided, which acquires characteristics information on the inside of an object using photoacoustic waves and that adjusts inrush currents at the time of power-on to enable a reduction in ampacity of a power source.

Further features of the present invention will become apparent from the following description of exemplary embodiments with reference to the attached drawings.

### BRIEF DESCRIPTION OF THE DRAWINGS

FIG. 1 is a diagram depicting a general configuration of the present invention;

FIG. 2 is a diagram depicting a connection configuration of a power source in the present invention;

FIG. 3 is a diagram illustrating a flow of operations at the time of power-on;

FIG. 4 is a diagram illustrating state transitions occurring at the time of power-on;

FIG. 5 is a timing diagram illustrating current values obtained at the time of power-on in conventional art;

FIG. 6 is a timing diagram illustrating current values obtained at the time of power-on in Embodiment 1;

FIG. 7 is a comparative diagram illustrating preparation times in a normal state in Embodiment 2;

FIG. 8 is a comparative diagram illustrating preparation times in a reactivation state in Embodiment 2; and

FIG. 9 is a timing diagram illustrating current values obtained at the time of power-on in Embodiment 3.

### DESCRIPTION OF THE EMBODIMENTS

Preferred embodiments of the present invention will be described below with reference to the drawings. However, dimensions, materials, shapes, relative arrangements, and the like of components described below should be changed as needed according to a configuration of an apparatus to which the present invention is applied and various conditions. Hence, the dimensions, materials, shapes, relative arrangements, and the like of the components described below are not intended to limit the scope of the present invention to the following description.

The present invention relates to a technique for detecting an acoustic wave propagating from an object to generate and acquire characteristics information on the interior of the object. Hence, the present invention is considered to be an object information acquiring apparatus or a method for controlling the object information acquiring apparatus, or an object information acquiring method or a signal processing method. The present invention is also considered as a program that allows an information processing apparatus including hardware resources, such as a CPU, to execute these methods, or a storage medium storing such a program.

The object information acquiring apparatus in the present invention includes an apparatus utilizing an acoustic tomography technique that involves irradiating an object with light (electromagnetic wave) and receiving (detecting) an acoustic wave propagating after being generated at a particular position inside the object or on a surface of the object. Such an object information acquiring apparatus may also be referred to as an imaging apparatus because the apparatus obtains characteristics information on the inside of the object in form of image data based on photoacoustic measurement.

The characteristics information in photoacoustic apparatuses indicates a source distribution for an acoustic wave resulting from light irradiation, an initial sound pressure distribution in the object, or an optical energy absorption density distribution or an absorption coefficient distribution derived from the initial sound pressure distribution, or a concentration distribution for substances forming a tissue. Specifically, the characteristics information is an oxidized and reduced hemoglobin concentration distribution, a blood component distribution such as an oxygen saturation distribution which is determined based on the oxidized and reduced hemoglobin concentration distribution, or a distribution of fat, collagen, or moisture. The characteristics information obtained may be distribution information on positions in the object instead of numerical data. That is, the object information may be distribution information such as an absorption coefficient distribution or an oxygen concentration distribution.

The term “acoustic wave” as used herein typically refers to an ultrasonic wave and includes an elastic wave referred to as a sound wave or an acoustic wave. An acoustic wave resulting from the photoacoustic effect is referred to as a photoacoustic wave or a photo-ultrasound wave. An electric signal into which the acoustic wave is converted by a probe or the like is referred to as an acoustic signal or a reception signal. However, the description of the ultrasonic wave or the acoustic wave herein is not intended to limit the wavelengths of these elastic waves. An electric signal (reception signal) derived from the photoacoustic wave is also referred to as a photoacoustic signal.

### Embodiment 1

### Apparatus Configuration

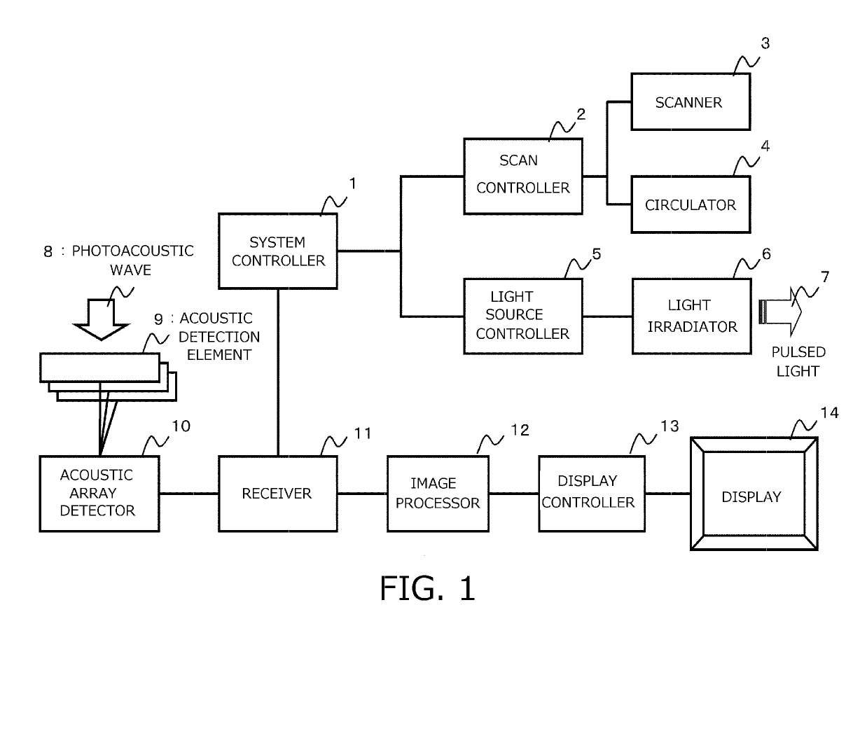

FIG. 1 is a diagram depicting a general configuration of the object information acquiring apparatus. A system controller serving to perform main control on the whole apparatus is denoted by reference numeral 1. A scan controller serving to control a scanning mechanism is denoted by reference numeral 2. A scanner that moves an acoustic array detector 10 to a predetermined position is denoted by reference numeral 3. A circulator that circulates a solution with which a space between the object and the acoustic array detector 10 is filled is denoted by reference numeral 4. Alight source controller that controls irradiation with pulsed light is denoted by reference numeral 5. A light irradiator that irradiates the object with pulsed light is denoted by reference numeral 6. The pulsed light is denoted by reference numeral 7. A photoacoustic wave is denoted by reference numeral 8. An acoustic detection element that detects the photoacoustic wave is denoted by reference numeral 9. The acoustic array detector to which a plurality of acoustic detection elements is attached is denoted by reference numeral 10. A receiver that loads reception signals detected by the acoustic detection elements is denoted by reference numeral 11. An image processor that calculates image data using reception signals of photoacoustic waves is denoted by reference numeral 12. A display controller that controls scan conversion and superimposed display of images is denoted by reference numeral 13. A display that displays image data is denoted by reference numeral 14. Various controllers can be implemented using a programmed computer or CPU.

First, the light source controller 5 irradiates the object with pulsed light 7 using the light irradiator 6. Subsequently, the acoustic array detector 10 including a plurality of acoustic detection elements 9 detects a photoacoustic wave 8 resulting from absorption, by a living tissue, of the energy of the pulsed light propagating and diffusing through the object, to output an electric signal. The receiver 11 executes signal processing on the electric signal. The image processor 12 then reconstructs the electric signal into image data to acquire characteristics information. Consequently, functional image data indicative of a substance distribution for the living tissue is obtained. The display controller 13 displays an image based on the image data on the display 14.

The scanner 3 may change the relative positional relation between the acoustic array detector 10 and the object and may move either the detector or the object. The present invention is also applicable to such a handheld apparatus as is gripped by a technician with the technician's hand and pressed against the object for measurement.

(Power Supply Connection Configuration)

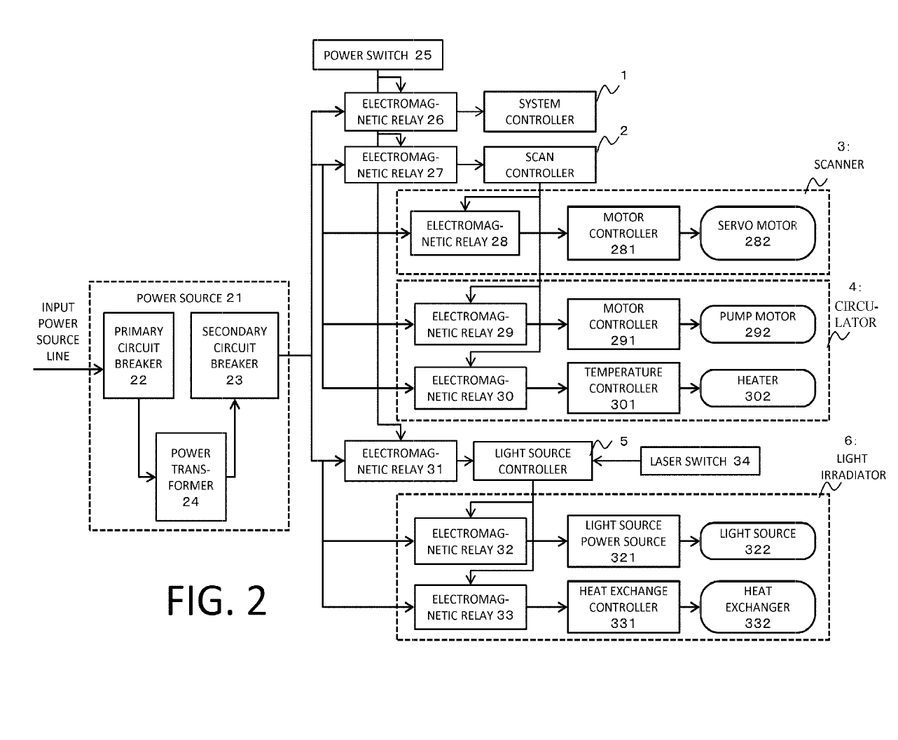

FIG. 2 is a diagram depicting a connection configuration of a power source in the present invention. A power source 21 includes a circuit breaker 22 connected to a primary side of an input power source line, a circuit breaker 23 connected to a secondary side of the input power source line, and a power transformer 24 providing a transformation function and an insulation function between the primary power source line and the secondary power source line. The transformation function of the power transformer 24 needs to keep the voltage of the secondary side connected to the apparatus constant even when the primary side has a different power source voltage due to a power source condition in a country or a district where the apparatus is installed. For example, in Japan, a single-phase 100 V/200 V power source is used on the primary side, whereas, in Europe, a single-phase 220 V-240 V power source is used on the primary side, with the secondary side fixed to, for example, 200 V. On the other hand, the insulation function of the power transformer 24 needs to be at a level that conforms with electrical safety standards for medical equipment. The safety standards specify, for example, the value of leakage current for means of operator protection (MOOP) and means of patient protection (MOPP).

A power switch 25 is a main switch for the whole apparatus. The power switch 25 performs direct ON/OFF control on electromagnetic relays 26, 27, and 31 to start supplying power to each of the system controller 1, the scan controller 2, and the light source controller 5, thus activating each of these units. The scan controller 2 can perform ON/OFF control on electromagnetic relays 28, 29, and 30. The light source controller 5 can perform ON/OFF control on electromagnetic relays 32 and 33. The power switch 25 is a manual switch but may be operated using an ON/OFF signal output from another control device. A method for controlling the switch and the relays is not limited to this. Any method may be used as long as the method allows power supply timings and activation timings for respective units to be controlled.

The scan controller 2 controls activation of the scanner 3 and the circulator 4. The electromagnetic relay 28 turns on and off a power source for a motor controller 281 and a servo motor 282 used to scan the acoustic array detector 10 included in the scanner 3 to a specified position. The electromagnetic relay 29 turns on and off a power source for a motor controller 291 and a pump motor 292 included in the circulator 4. The electromagnetic relay 30 turns on and off a power source for a heater 302 and a temperature controller 301.

A heater 302 used for the circulator 4 controls the liquid temperature of the solution in the matching layer so as to make the temperature of the solution equivalent to the temperature of the object. When the object is a living organism, the heater 302 adjusts the liquid temperature to a vicinity of the body temperature (for example, the temperature during bathing). The speed of sound in the solution varies with temperature, and thus, the liquid temperature is preferably uniformized as much as possible. The control of the heater 302 used for the circulator 4 may be adapted for a portion coming into contact with the object aside from the solution. Although the circulator 4 in this specification is described using the pump motor 292 and the motor controller 291, the circulator 4 may be implemented using any other type of pumps.

Activation of the light irradiator 6 is controlled by the light source controller 5. The light source controller 5 controls activation of a light source power source 321 supplying power to a light source 322 that generates pulsed light and a heat exchange controller 331 supplying power to a heat exchanger 332 that adjusts the ambient temperature of the light source 322. When the light source controller 5 is provided with a separate laser switch 34, the light irradiator 6 waits to be activated until the laser switch 34 is turned on after the power switch 25 is turned on. On the other hand, an apparatus configuration with the laser switch 34 not set therein need not be subjected to such a restraint.

The electromagnetic relay 32 turns on and off a power source for the light source 322 and the light source power source 321 included in the light irradiator 6. The electromagnetic relay 33 turns on and off a power source for the heat exchanger 332 and the temperature controller 301. In order to uniformize the amount of light and to make operations more stable, the heat exchanger 332 used for the light irradiator 6 is utilized to control the ambient temperature of the light source 322 so as to optimize light emission efficiency. The circulator 4 mainly performs heating control using the heater 302. On the other hand, for a crystal and a glass material used as working substances for a solid laser, a temperature needed for stable operation of the solid laser varies according to the types of the crystal and the glass material. Thus, both heating and cooling need to be controlled with the temperature of the solid laser compared with the ambient temperature.

(Process Flow)

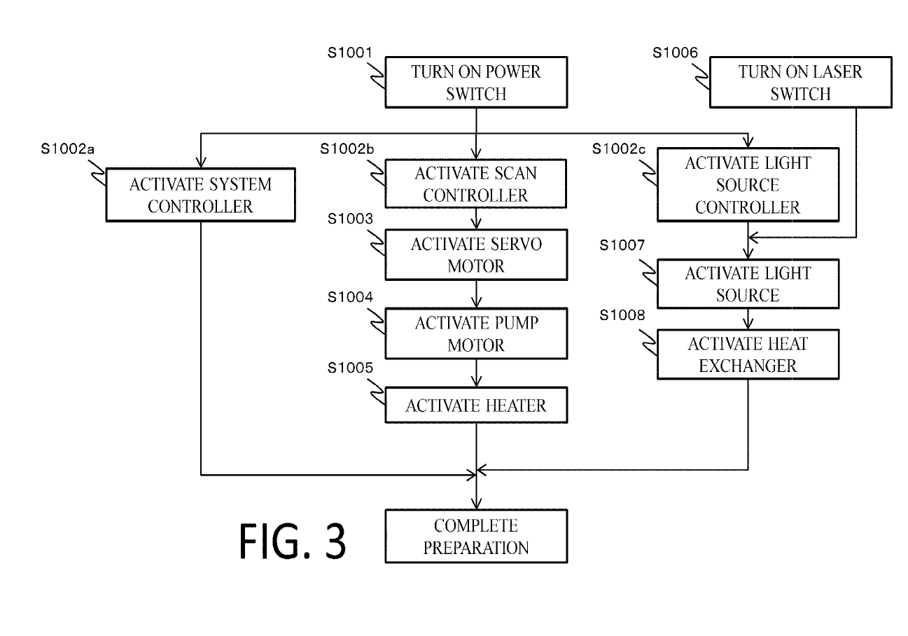

FIG. 3 is an operation flow of operations performed when the apparatus is powered on. First, in step S1001, the power switch 25 is turned on. Then, in steps S1002a to S1002c, the system controller 1, the scan controller 2, and the light source controller 5, respectively, are activated.

The devices in the scanner 3 have activation timings therefor controlled in accordance with instructions from the scan controller 2. In step S1003, the servo motor is activated. In step S1004, the pump motor is activated. In step S1005, the heater is activated. Activation timings for the scanner 3 are managed by the scan controller 2.

The devices in the light irradiator 6 have activation timings therefor controlled in accordance with instructions from the light source controller 5. In step S1006, when the laser switch is determined to have been turned on, the light source is activated in step S1007, and the heat exchanger is activated in step S1008. Activation timings for the light irradiator 6 are managed by the light source controller 5.

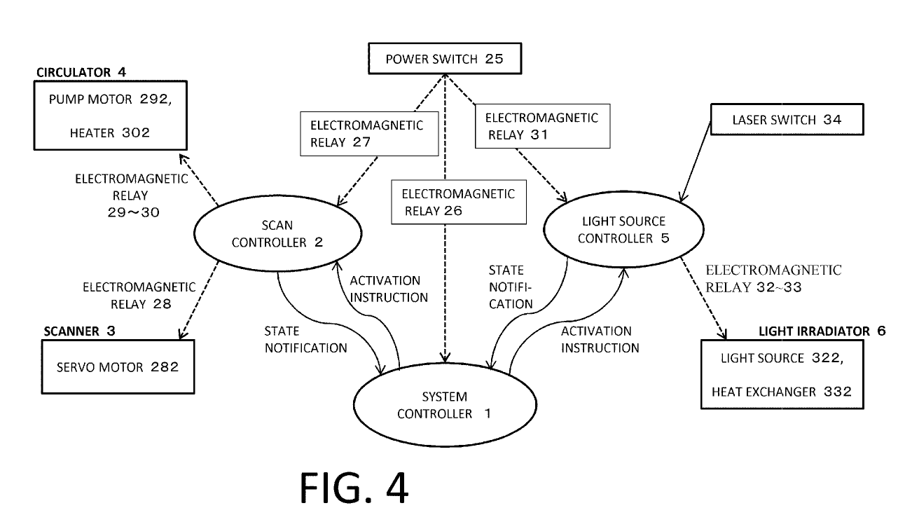

FIG. 4 is a diagram illustrating state transitions occurring at the time of power-on. First, the power switch is turned on to turn on the electromagnetic relays 26, 27, and 31 to start-up the system controller 1, the scan controller 2, and the light source controller 5. Although not depicted in the drawings, the system controller 1 has communication means for both the scan controller 2 and the light source controller 5 and can thus obtain state notifications from and give activation instructions to both the scan controller 2 and the light source controller 5. That is, the system controller 1 is a main controller that can adjust an activation order and activation timings for the whole apparatus.

The scan controller 2 adjusts activation timings for the scanner 3 and the circulator 4 in accordance with instructions from the system controller 1. The light source controller 5 monitors the state of the laser switch 34 to give state notification and adjusts an activation timing for the light irradiator 6 in accordance with an instruction from the system controller 1.

(Control Timings)

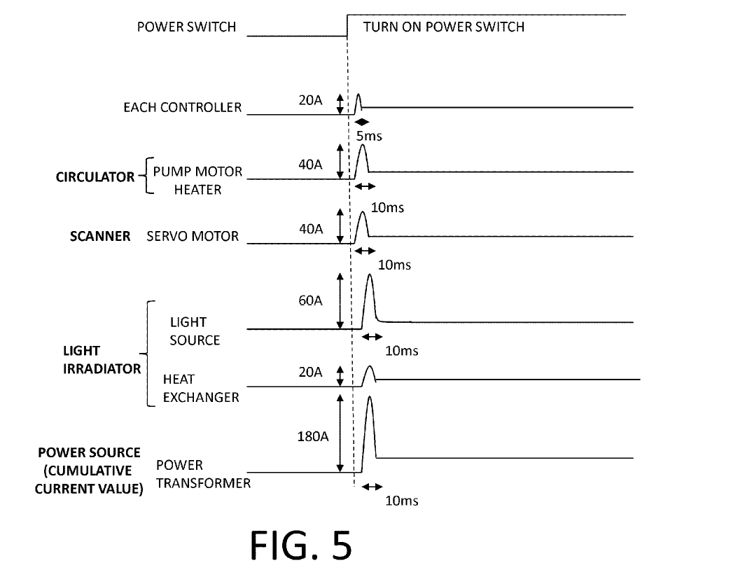

FIG. 5 is a timing diagram illustrating current values obtained at the time of power-on in the related art. FIG. 5 illustrates current values obtained when all blocks are simultaneously started up with activation timings for inrush currents not adjusted. In this case, the sum of inrush currents flowing to the secondary side of the power source is 180 A, resulting in a large current flowing in a short time of 10 ms. Thus, the power source needs to include power source components adapted for this current value. Consequently, the power source needs to have a capacity of 180 A.

If the actually flowing current is larger than the rated capacity of the power transformer, when inrush currents flow, a voltage drop occurs to make the power source voltage equal to or smaller than a specified value. Then, the connected devices are precluded from being correctly activated. On the other hand, if the capacity of the power transformer is increased so as to be adapted for the sum of the inrush currents, the voltage drop problem can be suppressed, whereas the power transformer has an increased external size and an increased weight. This is disadvantageous in installation space and apparatus manufacturing costs.

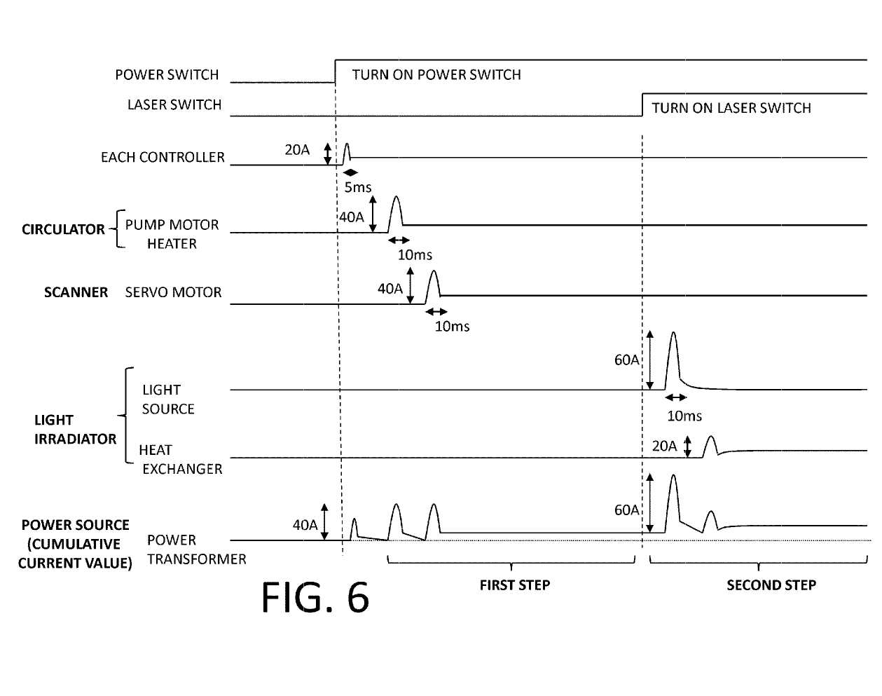

FIG. 6 is a timing diagram illustrating current values obtained at the time of power-on in the present embodiment, in other words, the values of currents flowing through the devices at the time of power-on. First, the power switch is used to activate the controllers. The current value obtained at this time is 20 A. Subsequently, in a first step, the pump motor and the heater in the circulator and the servo motor 282 in the scanner 3 are activated by the scan controller 2 with the activation timings for the pump motor and the heater, and the servo motor 282 adjusted. For all of these devices, the current value is 40 A, and thus, only up to 40 A of inrush currents flow through the power source 21. A steady current value for each of the controllers, the circulator, and the scanner is much smaller than a peak value during activation. Given that the steady current value of each device is estimated to be 10% of the peak value during activation, the maximum current obtained when the inrush current flows through the scanner is only 46 A.

At a second step, the activation timing for the light irradiator 6 following turn-on of the laser switch 34 is illustrated. At this time, the activation timings for the light source 322 and the heat exchanger 332 are adjusted to set the maximum value of the inrush current flowing through the power source to 60 A. In the first step and the second step as described above, the maximum value of the cumulative current value of the power source is 60 A. Thus, the activation timing for the inrush current is adjusted to allow the ampacity of the power source to be reduced from 180 A to 60 A. As a result, the external size and weight of the power source can be significantly reduced. When the light irradiator is started up without stopping already activated devices, the steady current flowing through each of the devices needs to be taken into account. In that case, the ampacity is equal to 60 A plus the value of the steady current.

The order of the activation timings in FIG. 6 is an example. The object of the present invention can be accomplished as long as the activation timings for the devices can be controlled so as to prevent the current peaks from concentrating within a short time. For example, the order of the first and second steps may be changed.

### Effects

As described above, in the photoacoustic imaging apparatus in the present embodiment, the ampacity of the power source can be reduced by adjusting the activation timings for the devices at the time of power-on. This in turn enables a reduction in the ampacity of a distribution board at an installation site and in the capacity of the power transformer 24 in the power source 21, that is, a reduction in size and weight.

### Preferred Configuration Example

Examples of the components of the apparatus will be described. The system controller 1, the scan controller 2, the light source controller 5, the image processor 12, the display controller 13, and the like can be implemented using an information processing apparatus (a PC, a workstation, or the like) including a processor operating in accordance with programs, a memory, and an I/O interface. The functions may be implemented by respective individual process circuits. Alternatively, a single information processing apparatuses may be used or a number of information processing apparatuses may be used which are connected to a network to operate in cooperation with one another. For the image reconstructing process executed by the image processor 12, various known techniques (phasing addition, back projection, Fourier transform, and the like) may be utilized. Information on preferred activation control can be saved in the memory. Examples of the information on preferred activation control include fixed values for priorities for activation and information on power consumption needed to actively set the activation order.

As the light source, a laser apparatus capable of providing a large capacity output is preferably used. However, a light-emitting diode or a flash lamp may be used as the light source. The use of a wavelength-variable light source enables measurement utilizing a variation in absorbance according to wavelength (for example, enables oxygen saturation to be acquired). For an optical system that guides light to the object, optical members such as optical fibers, mirrors, prisms, lenses, and the like may be utilized.

Each of the acoustic detection elements 9 converts a received acoustic wave into an electric signal and outputs the electric signal. For example, piezoelectric elements, capacitive elements, or a Fabry-Perot interferometer may be used as the acoustic detection elements 9. For the acoustic array detector 10, a metal or a resin having a predetermined strength may be suitably utilized in order to stably support the elements. The elements are arranged in a linear form, a planar form, a sparse array form, or the like according to a measurement requirement. Preferably, the elements are arranged on an inner surface of a bowl-shaped acoustic array detector 10 so as to provide a high-sensitivity region in which directions of the elements in which the elements exhibit high reception sensitivity (directional axes) concentrate. The receiver 11 includes a circuit that executes an amplification process, a digital conversion process, or a correction process on an analog electric signal output by each of the elements, as needed. Alternatively, the elements may be arranged on a highly portable handheld probe.

The scanner 3 includes a stage, a stepping motor, or the like and two- or three-dimensionally moves at least one of the object and the acoustic array detector 10 to change the relative positional relation between the object and the acoustic array detector 10. This operation of the scanner 3 is effective for allowing a large area of interest of the object to be measured and receiving acoustic waves in various directions to improve reconstruction accuracy and an SN ratio. The scan controller 2 acquires the position of the scanner and transmits a control signal to the scanner to allow the scanner to move on a predetermined trajectory. For example, when the object is held under pressure by a plate-like holding member, two-dimensional scanning is suitable in which the main scanning and sub-scanning are performed along the plate. When a cup-shaped holding member is used to hold the object and a bowl-shaped detector is used, spiral scanning is used. The circulator 4 includes a container in which a matching liquid such as water or castor oil is stored, a pipe through the matching liquid is circulated, and a temperature adjusting mechanism.

As the display 14, any display such as a liquid crystal apparatus or an organic EL apparatus may be used. The display 14 may be a part of the apparatus in the present invention or an external apparatus that receives and displays image data via an interface. The image processor 12 or the image processor 12 and the receiver 11 correspond to an information processor in the present invention. The system controller 1 corresponds to a power source controller in the present invention. The acoustic detection elements 9 or the acoustic detection elements 9 and the acoustic array detector 10 correspond to a detector in the present invention.

### Embodiment 2

In Embodiment 1, fixed values are set for the priorities for activation of the devices. In the present embodiment, the priorities for activation are changed according to an apparatus status.

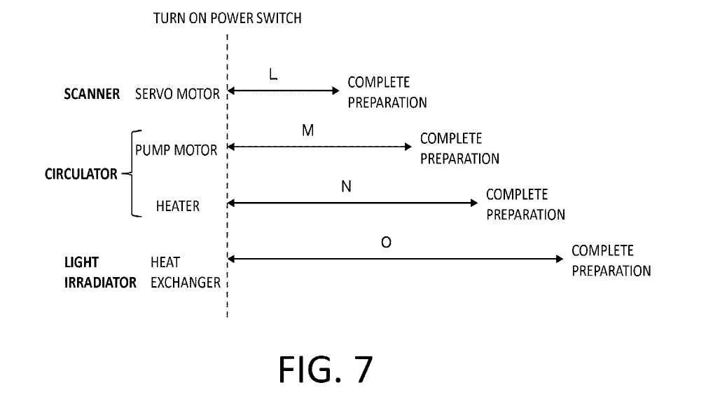

First, a case will be described where a high priority is given to activation of the controller with a long preparation time. FIG. 7 illustrates, for each of the devices included in the apparatus, that is, the scanner 3, the circulator 4, and the light irradiator 6, a general preparation time after the device is powered on to start operation and before the device actually gets prepared for measurement.

The preparation time for the servomotor 282 in the scanner 3 includes a time needed to move the position of the acoustic array detector 10 to a home position during activation of the apparatus. Thus, the preparation time varies according to the position of the acoustic array detector 10 at the time of power-off. That is, given that it is possible to determine the position in which the detector is placed at the time of activation or the position where the detector is stopped after the last measurement, the preparation time can be set to be the time L needed to move to the home position. The detector position can be acquired by the scan controller 2 by referencing apparatus parameters or referencing a memory in which the position where the detector is stopped after the last measurement is recorded.

The pump motor 292 in the circulator 4 needs await time until the matching layer is filled with a desired amount of solution. The wait time varies according to the amount of solution remaining between the object and the acoustic array detector 10 at the time of power-off. That is, given that the amount of solution remaining at the time of power-off can be determined, the preparation time can be set to be a time M needed to fill the matching layer with the desired amount of solution based on the amount of solution remaining at the time of power-off. The amount of solution can be acquired using a water level sensor or the like.

The heater 302 in the circulator 4 needs a wait time until the temperature of the solution is elevated to a specified value (set value). This preparation time varies according to the temperature at the time of activation of the apparatus, the temperature at the time of power-off, an elapsed time after power-off, and the like. Given that the temperature can be monitored at the time of activation, a time needed to elevate the temperature of the solution to a set value can be estimated. Thus, the estimated time is set to be a preparation time N.

The light irradiator 6 needs a wait time for which the heat exchanger 332 is operated to adjust the ambient temperature of the light source to a set value. This preparation time varies according to the temperature of the heat exchanger 332 at the time of power-off. Given that the temperature of the heat exchanger 332 can be monitored at the time of activation, a time needed to elevate the ambient temperature to a set value can be estimated. Thus, the estimated time is set to be a preparation time O.

As depicted in a state transition diagram in FIG. 4 illustrating state transitions occurring at the time of activation, the scan controller 2 and the light source controller 5 give state notifications to the system controller 1. The state notifications include information on the preparation times L to O. The notification from the servo motor 282 to the system controller 1 is indicative of the time L. The notification from the pump motor 292 to the system controller 1 is indicative of the time M. The notification from the heater 302 to the system controller 1 is indicative of the time N. The notification from the heat exchanger 332 to the system controller 1 is indicative of the time O.

The system controller 1 compares the preparation times L to O with one another and starts activation by giving a top priority to the longest preparation time. As a result, the wait time until measurement can be started is reduced. In a comparison of wait times in a normal state illustrated in FIG. 7, the length of the preparation time is in the order of O>N>M>L, and thus, the heat exchanger 332, the heater 302, the pump motor 292, and the servo motor 282 may start to be activated in this order.

### Variations

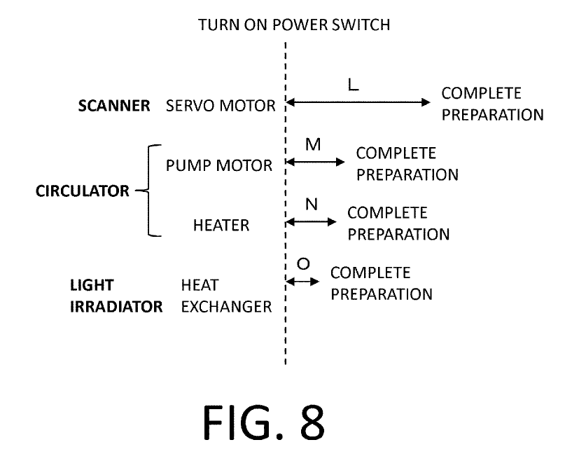

In actuality, each of the wait times varies according to an initial state, and the priorities are preferably changed according to status. FIG. 8 is a diagram illustrating wait times in a state where the apparatus power source is reactivated. In this case, the power-off period is short, and thus, the preparation times N and O for the temperature control performed by the heater 302 and the heat exchanger 332 are very short. On the other hand, the servo motor 282 and the pump motor 292 need relatively long preparation times depending on the position where the detector is stopped and the height of the surface of the solution. In FIG. 8, the length of the preparation time is in the order of L>M>N>O, the servo motor 282, the pump motor 292, the heater 302, and the heat exchanger 332 start to be activated in this order.

As described above, in the photoacoustic imaging apparatus in the present embodiment, the priorities for activation are flexibly changed in view of changes in the preparation time for each device according to the state thereof. That is, devices needing longer preparation times are activated earlier, with activation of the other devices delayed. As a result, inrush current peaks in the respective devices at the time of device start-up are shifted from one another, enabling a reduction in the ampacity of the power source. This in turn enables a reduction in the ampacity of the distribution board at the installation site and in the capacity of the power transformer 24 in the power source 21, that is, a reduction in size and weight.

Compared to Embodiment 1, the present embodiment enables a reduction in preparation time. The present embodiment is also effective for enabling a further reduction in preparation time by actively controlling the activation start time of each device according to the state of the device at the time of power-on. In the present embodiment, the priorities are determined based on the length of the preparation time. However, the priorities may alternatively be determined based on the method described in Embodiment 1. This enables time intervals between a plurality of measurements to be reduced to make the measurement more efficient even when the scanning photoacoustic apparatus includes a laser in order to achieve a high output.

Alternatively, the priorities of device activation may be determined using the values of times that are needed, for example, to elevate and stabilize the temperature, to raise the solution level, and to move the position of the detector, and that are estimated based on information obtained from sensors corresponding to the devices (a temperature sensor, a water gauge, a position sensor, and the like). At the end of the last measurement, the solution level and the position information may be saved to the memory. Moreover, for the temperature, the degree of a decrease in temperature may be estimated based on a time having elapsed since the end of the last measurement.

### Embodiment 3

In Embodiment 1, the timing control is performed in association with the inrush currents at the time of power-on. However, for devices with power consumption thereof fluctuating significantly during operation, control of an operating current needs to be performed in conjunction with the timing control for the inrush currents.

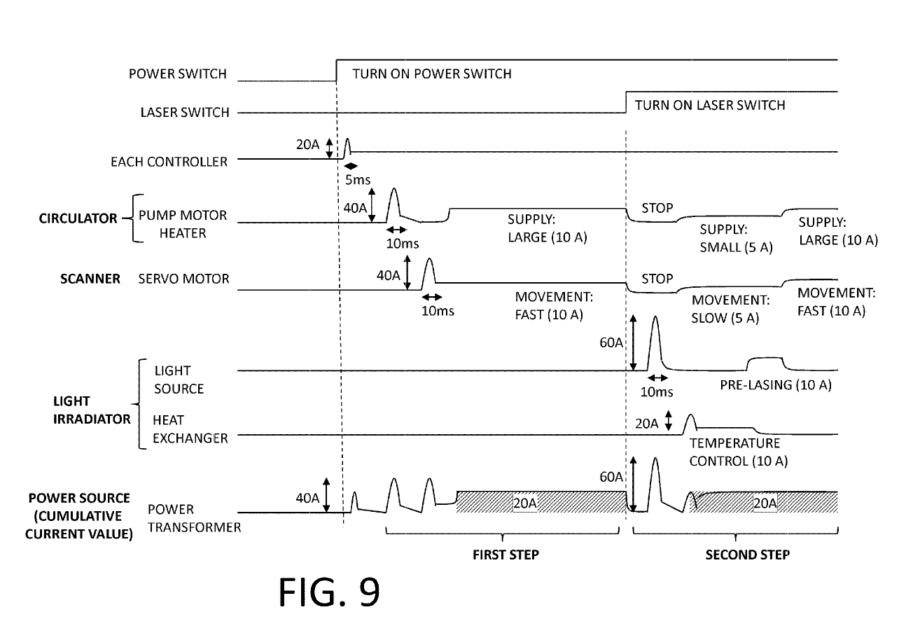

FIG. 9 is a timing diagram for Embodiment 3 illustrating the values of currents flowing through the devices after turn-on of the power switch 25, in addition to the values of the currents flowing at the time of power-on. At a first step, the activation timing for the scan controller 2 after turn-on of the power switch 25 is illustrated. At a second step, the activation timing for the light irradiator 6 after turn-on of the laser switch 34 is illustrated.

For the pump motor 292 in the circulator 4, the wait time until measurement is started can be reduced by increasing a speed at which the solution is fed at an initial stage of activation. Thus, the amount of solution fed is increased, leading to a steady flow of a consumption current of 10 A even after activation of the device.

For the servomotor 282 in the scanner 3, the position of the acoustic array detector 10 needs to be moved to the home position at the initial stage of activation. Therefore, an attempt to perform a quick moving scan in order to reduce the wait time results in a steady flow of a current of 10 A even after activation of the device.

The device in the light irradiator 6 is activated by the laser switch 34. An inrush current of 60 A flows through the light source. Since a steady operating current of 20 A flows through the circulator 4 and the scanner 3 as described above, the power source 21 needs to have a capacity large enough to allow for a current of 60 A+20 A=80 A.

Thus, in the present embodiment, at the timing to start activate the light source 322, the operation of the pump motor 292 and the servo motor 282 is stopped to allow an inrush current to flow only through the light source 322. This enables the maximum current to be limited to 60 A. An inrush current of 20 A flows through the heat exchanger 332 when the heat exchanger 332 is activated, and the operating current flowing through the heat exchanger 332 is 10 A. Thus, the pump motor 292 and the servo motor 282 can be concurrently operated while the heat exchanger 332 is in operation.

On the other hand, the operating current is the total value of currents steadily flowing though the power source. The circuit breaker in the power source 21 is used to prevent the operating current from becoming excessive. The circuit breaker has operational characteristics designed to prevent unwanted tripping of the circuit breaker resulting from a short-time flow of an excess current such as an inrush current flowing at the time of power-on or a motor starting current. Thus, the ampacity of the circuit breaker is calculated from the total value of the actual operating currents.

The maximum current value of the operating current is 10 A for each of the pump motor 292, the servo motor 282, the light source 322, and the heat exchanger 332. Hence, when these devices are fully operated, the maximum value of the steady currents is 40 A. However, when the total value of the operating currents, for which the maximum value is 40 A, is reduced to 20 A or less by controlling the operation of the devices, the secondary circuit breaker 23 can be selected to have a rated ampacity of 20 A.

As described above, in the photoacoustic imaging apparatus in the present embodiment, devices other than those which are preferentially operated are reduced in operating current to enable a reduction in the ampacity of the power source. This in turn enables a reduction in the ampacity of the distribution board at the installation site and in the capacity of the power transformer 24 in the power source 21, that is, a reduction in size and weight. Furthermore, in the present embodiment, the activation start time for each device is controlled in view of the magnitude of the operating current flowing through the device. This serves to further reduce the capacity of the transformer.

In the present embodiment, the priorities for activation of the devices may be determined in accordance with the method described in Embodiment 1 and in which the devices are activated in the order preset using fixed values. Alternatively, the priorities for activation of the devices may be determined in accordance with the method described in Embodiment 2 and in which the priorities for the activation are changed according to the apparatus status.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment (s) of the present invention, and by a method performed by the computer of the system or apparatus by, for example, reading out and executing the computer executable instructions from the storage medium to perform the functions of one or more of the above-described embodiment (s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-101931, filed on May 19, 2015, which is hereby incorporated by reference herein in its entirety.

What is claimed is:

1. An object information acquiring apparatus comprising:
alight source controller that controls radiation of light from a light source;
a detector that detects an acoustic wave generated by an object irradiated with the light;
an information processor that acquires characteristics information on an inside of the object using the acoustic wave;
a scan controller that changes a relative position between the detector and the object,
a power source that supplies electricity for use by the object information acquiring apparatus; and
a power source controller that controls activation timings for the light source controller and the scan controller so as to prevent a current value of a current flowing through the object information acquiring apparatus from exceeding an ampacity of the power source.

2. The object information acquiring apparatus according to claim 1, further comprising a circulator that controls a temperature of a matching liquid that acoustically couples the object and the detector together,
wherein the power source controller further controls an activation timing for the circulator.

3. The object information acquiring apparatus according to claim 1, wherein the power source controller has priorities for activation of the light source controller and the scan controller as fixed values.

4. The object information acquiring apparatus according to claim 2, wherein the power source controller has priorities for activation of the light source controller, the scan controller, and the circulator as fixed values.

5. The object information acquiring apparatus according to claim 1, wherein the power source controller activates devices in an order of the circulator, the scan controller, and the light source controller.

6. The object information acquiring apparatus according to claim 1, wherein the power source controller preferentially activates whichever one of the light source controller and the scan controller that has a longer preparation time.

7. The object information acquiring apparatus according to claim 2, wherein the power source controller activates the light source controller, the scan controller, and the circulator in order of decreasing preparation time.

8. The object information acquiring apparatus according to claim 7, wherein the power source controller acquires a temperature of the light source, a temperature of the matching liquid, a level of the matching liquid, and a position of the detector in use of sensors and estimates the preparation times in accordance with outputs from the sensors.

9. The object information acquiring apparatus according to claim 1, wherein the power source controller reduces operating currents flowing through devices that are not a device to be activated.

10. The object information acquiring apparatus according to claim 1, further comprising a display controller that controls display of the characteristics information on the object on a display,
wherein the power source controller further controls an activation timing for the display controller.
